SEQUENCE LISTING (54 sequences)

SEQ ID NO: 1 - protein, 8 aa, Artificial Sequence
  Synthetic peptide sequence FLAG epitope
DYKDDDDK

SEQ ID NO: 2 - protein, 10 aa, Artificial Sequence
  Synthetic peptide sequence Myc epitope
EQKLISEEDL

SEQ ID NO: 3 - protein, 9 aa, Artificial Sequence
  Synthetic peptide sequence HA epitope
YPYDVPDYA

SEQ ID NO: 4 - protein, 6 aa, Artificial Sequence
  Synthetic peptide sequence 6x His epitope
HHHHHH

SEQ ID NO: 5 - protein, 11 aa, Artificial Sequence
  Synthetic peptide sequence T7 epitope tag
MASMTGGQQMG

SEQ ID NO: 6 - protein, 7 aa, Artificial Sequence
  Synthetic peptide sequence (the heptad unit of acid (En) coiled coils)
VSALEKE

SEQ ID NO: 7 - protein, 7 aa, Artificial Sequence
  Synthetic peptide sequence (the heptad unit of base (Kn) coiled coils)
VSALKEK

SEQ ID NO: 8 - DNA, 105 bp, Artificial Sequence
  Synthetic nucleotide sequence coding for acid E5 coiled coils
gaagtttctgctttggaaaaggaagtttctgctttggaaaaggaagtttctgccttagagaaagaggtctccgctttggaaaaggaagtttctgctttggaaaag

SEQ ID NO: 9 - DNA, 84 bp, Artificial Sequence
  Synthetic nucleotide sequence coding for acid E4 coiled coils
gaagtttctgctttggaaaaggaagtttctgccttagagaaagaggtctccgctttggaaaaggaagtttctgctttggaaaag

SEQ ID NO: 10 - DNA, 63 bp, Artificial Sequence
  Synthetic nucleotide sequence coding for acid E3 coiled coils
gaagtttctgccttagagaaagaggtctccgctttggaaaaggaagtttctgctttggaaaag

SEQ ID NO: 11 - DNA, 105 bp, Artificial Sequence
  Synthetic nucleotide sequence coding for base K5 coiled coils
aaggtttctgctttgaaggaaaaggttagtgcattaaaagagaaagtctccgctttgaaggaaaaggtttctgctttgaaggaaaaggtttctgctttgaaggaa

SEQ ID NO: 12 - DNA, 84 bp, Artificial Sequence
  Synthetic nucleotide sequence coding for base K4 coiled coils
aaggtttctgctttgaaggaaaaggttagtgcattaaaagagaaagtctccgctttgaaggaaaaggtttctgctttgaaggaa

SEQ ID NO: 13 - DNA, 63 bp, Artificial Sequence
  Synthetic nucleotide sequence coding for base K3 coiled coils
aaggtttctgctttgaaggaaaaggttagtgcattaaaagagaaagtctccgctttgaaggaa

SEQ ID NO: 14 - DNA, 267 bp, Artificial Sequence
  Synthetic nucleotide sequence coding for alpha 1 mating type factor signal sequence
atgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagct

SEQ ID NO: 15 - DNA, 54 bp, Artificial Sequence
  Synthetic nucleotide sequence coding for Aga2 signal sequence
atgcagttacttcgctgtttttcaatattttctgttattgcttcagttttagca

SEQ ID NO: 16 - protein, 89 aa, Artificial Sequence
  Synthetic peptide sequence (alpha 1 mating type factor signal sequence)
MRFPSIFTAVLFAASSALAAPVNTTTEDETAQIPAEAVIGYSDLEGDFDVAVLPFSNSTNNGLLFINTTIASIAAKEEGVSLEKREAEA

SEQ ID NO: 17 - protein, 18 aa, Artificial Sequence
  Synthetic peptide sequence (Aga2 signal sequence)
MQLLRCFSIFSVIASVLA

SEQ ID NO: 18 - DNA, 261 bp, Artificial Sequence
  Synthetic nucleotide sequence coding for Aga2
atgcagttacttcgctgtttttcaatattttctgttattgcttcagttttagcacaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgttttt

SEQ ID NO: 19 - protein, 87 aa, Artificial Sequence
  Synthetic polypeptide sequence Aga2
MQLLRCFSIFSVIASVLAQELTTICEQIPSPTLESTPYSLSTTTILANGKAMQGVFEYYKSVTFVSNCGSHPSTTSKGSPINTQYVF

SEQ ID NO: 20 - DNA, 462 bp, Artificial Sequence
  Synthetic nucleotide sequence coding for NeGFP
gtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggcc

SEQ ID NO: 21 - protein, 154 aa, Artificial Sequence
  Synthetic polypeptide sequence NeGFP
VSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMA

SEQ ID NO: 22 - DNA, 252 bp, Artificial Sequence
  Synthetic nucleotide sequence coding for CeGFP
gacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

SEQ ID NO: 23 - protein, 84 aa, Artificial Sequence
  Synthetic polypeptide sequence CeGFP
DKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 24 - DNA, 664 bp, Artificial Sequence
  Synthetic nucleotide sequence (GAL1-GAL10 bi-directional promoter)
ttcttactttttttttggatggacgcaaagaagtttaataatcatattacatggcattaccaccatatacatatccatatacatatccatatctaatcttacttatatgttgtggaaatgtaaagagccccattatcttagcctaaaaaaaccttctctttggaactttcagtaatacgcttaactgctcattgctatattgaagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatc

SEQ ID NO: 25 - DNA, 385 bp, Artificial Sequence
  Synthetic nucleotide sequence (GAL1 promoter)
accggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccgg

SEQ ID NO: 26 - DNA, 269 bp, Artificial Sequence
  Synthetic nucleotide sequence (GAL10 promoter)
ccggtgaagacgaggacgcacggaggagagtcttccttcggagggctgtcacccgctcggcggcttctaatccgtacttcaatatagcaatgagcagttaagcgtattactgaaagttccaaagagaaggtttttttaggctaagataatggggctctttacatttccacaacatataagtaagattagatatggatatgtatatggtggtaatgccatgtaatatgattattaaacttctttgcgtccatccaaaaaaaaagtaagaa

SEQ ID NO: 27 - protein, 5 aa, Artificial Sequence
  Synthetic peptide sequence Glycine linker sequence
GGGGS

SEQ ID NO: 28 - DNA, 82 bp, Artificial Sequence
  Synthetic oligonucleotide sequence forward primer for E5
agctgctagcgaagtttctgctttggaaaaggaagtttctgctttggaaaaggaagtttctgccttagagaaagaggtctcc

SEQ ID NO: 29 - DNA, 64 bp, Artificial Sequence
  Synthetic oligonucleotide sequence reverse primer for E5
caccactagtcttttccaaagcagaaacttccttttccaaagcggagacctctttctctaaggc

SEQ ID NO: 30 - DNA, 88 bp, Artificial Sequence
  Synthetic oligonucleotide sequence forward primer for K5
caccatcgatttccttcaaagcagaaaccttttccttcaaagcagaaaccttttccttcaaagcggagactttctcttttaatgcact

SEQ ID NO: 31 - DNA, 91 bp, Artificial Sequence
  Synthetic oligonucleotide sequence reverse primer for K5
ctcgacgcgtggtggtggtggttctggtggtggtggttctaaggtttctgctttgaaggaaaaggttagtgcattaaaagagaaagtctcc

SEQ ID NO: 32 - DNA, 30 bp, Artificial Sequence
  Synthetic oligonucleotide sequence forward primer for LFA-1 I domain
ttgtgtcgacaacgtagacctggtatttct

SEQ ID NO: 33 - DNA, 26 bp, Artificial Sequence
  Synthetic oligonucleotide sequence reverse primer for LFA-1 I domain
ccatggagtcaggtcctgtttgcttg

SEQ ID NO: 34 - DNA, 30 bp, Artificial Sequence
  Synthetic oligonucleotide sequence forward primer for AL57
ggatgctagcgaagttcaattgttagagtc

SEQ ID NO: 35 - DNA, 29 bp, Artificial Sequence
  Synthetic oligonucleotide sequence reverse primer for AL57
ggtcggatccagttcgtttgatttccacc

SEQ ID NO: 36 - DNA, 30 bp, Artificial Sequence
  Synthetic oligonucleotide sequence forward primer for TS1/22
agcagctagccaggttcacctgcagcaatc

SEQ ID NO: 37 - DNA, 29 bp, Artificial Sequence
  Synthetic oligonucleotide sequence reverse primer for TS1/22
caccactagtagcccgtttcagctccagc

SEQ ID NO: 38 - DNA, 34 bp, Artificial Sequence
  Synthetic oligonucleotide sequence forward primer for BoNT/A-LC
cgttgtcgaccaattcgttaacaagcagttcaac

SEQ ID NO: 39 - DNA, 34 bp, Artificial Sequence
  Synthetic oligonucleotide sequence reverse primer for BoNT/A-LC
agtcccatggtttgctcgttataatgcctctaac

SEQ ID NO: 40 - DNA, 31 bp, Artificial Sequence
  Synthetic oligonucleotide forward primer for BoNT/B-LC
cgttgtcgacccggtgacgatcaacaacttc

SEQ ID NO: 41 - DNA, 33 bp, Artificial Sequence
  Synthetic oligonucleotide sequence reverse primer for BoNT/B-LC
agtcccatggaacgctcttacacatttggattt

SEQ ID NO: 42 - DNA, 35 bp, Artificial Sequence
  Synthetic oligonucleotide sequence forward primer for VHH-B8 andG6
agctgctagcgcatgccaggctcatgtccagctgc

SEQ ID NO: 43 - DNA, 39 bp, Artificial Sequence
  Synthetic oligonucleotide sequence reverse primer for VHH-B8 andG6
gttcggatccactagttggttgtggttttggtgtcttgg

SEQ ID NO: 44 - DNA, 34 bp, Artificial Sequence
  Synthetic oligonucleotide sequence forward primer for VHH library
agctgctagccagrctsagktgcagctcgtggag

SEQ ID NO: 45 - DNA, 31 bp, Artificial Sequence
  Synthetic oligonucleotide sequence reverse primer I for VHH library
tggtggatccttgtggttttggtgtcttggg

SEQ ID NO: 46 - DNA, 30 bp, Artificial Sequence
  Synthetic oligonucleotide sequence reverse primer II for VHHlibrary
tggtggatccggggtcttcgctgtggtgcg

SEQ ID NO: 47 - protein, 133 aa, Artificial Sequence
  Synthetic polypeptide sequence VHH1a
QTEVQLVESGGALVQPGGSLTLSCVSSGSSLDDYAIGWFRQAPGKDRIGAPCIGSDGSATNYVGDASYRFTISREKNTVYLQMDSLKTDDTAVYSCAAYSTGWGPNCESLDGTVAWGKGTPVTVTSAHHSEDP

SEQ ID NO: 48 - protein, 134 aa, Artificial Sequence
  Synthetic polypeptide sequence VHH48
QAELQLVESGGGSVHPGESLRLSCAGSGSLFGYYAIGWFRQAPGKECEGVSCLSPNGDETRYASSVKDRFIISRDNAKNTVYLQMNSLKPEDTGVYYCADDPCSSGIWGSLPKCYSAGQGTEVTVSSAHHSEDP

SEQ ID NO: 49 - protein, 133 aa, Artificial Sequence
  Synthetic polypeptide sequence VHH57
QAEVQLVESGGGLVEPGGSLSLSCTASEFFLDFYAVAWFRQAPGKEREGISCISGTDASADYKDSVKGRFTISRDDAKGTVYLQMMNLKPEDTAVYYCAAPADCGCSGYMCDNRYWGQGTQVNVSSAHHSEDP

SEQ ID NO: 50 - protein, 145 aa, Artificial Sequence
  Synthetic polypeptide sequence VHH41
QTQVQLVESGGGLVQPGGSLNLSCSASGGTFYYAIGWFRQAPGKEREEVSCISSSGGSPDYSDSVKGRFTTSRDVAKKTVYLQMNSLRPEDTALYQCATVRPGPQCSGYVCQDTCAFGSWGQGTQVTVSSGESSTVSSAHHSEDP

SEQ ID NO: 51 - protein, 133 aa, Artificial Sequence
  Synthetic polypeptide sequence G6
QAHVQLQQSGGGLVQPGGSLRLSCAASGFTLDYYAIGWFRQAPGKEREGVSCISSSGGSTYYADSVKGRFTISRDNAKNTVYLQMNSLKPEDTAVYYCAVAQSCLRGGVRGGYWGQGTQVTVSSEPKTPKPQP

SEQ ID NO: 52 - protein, 132 aa, Artificial Sequence
  Synthetic polypeptide sequence B8
QAHVQLQQSGGGLVQPGGSLRLSCAASGSIFSIYAMGWYRQAPGKQRELVAAISSYGSTNYADSVKGRFTISRDNAKNTVYLQMNSLKPEDTAVYYCNADIATMTAVGGFDYWGQGTQVTVSSEPKTPKPQP

SEQ ID NO: 53 - protein, 135 aa, Artificial Sequence
  Synthetic polypeptide sequence B10
QLQLVESGGGMVQPGGSLRLSCAASGFTFSTYDMSWVRQAPGKGPEWVSIINAGGGSTYYAASVKGRFAISRDNAKNTLYLQMNNLKPEDTALYYCARVASYYCRGYVCSPPEFDYWGQGTQVTVSSEPKTPKPQ

SEQ ID NO: 54 - protein, 129 aa, Artificial Sequence
  Synthetic polypeptide sequence C3
QVQLVESGGGLVQPGDSLTLSCAVSGFSFSSYKMSWVRQTPGKGLEWVSSINSGGGTTNYADSVKGRFTISRDNAKNMLYLQMNKLKPEDTAVYYCAHRVVTGQLDLYDYWGQGTQVTVSSEPKTPKPQ

(19) United States
(12) Patent Application Publication
(10) Pub. No.: US 2010/0075326 A1
(43) Pub. Date: Mar. 25, 2010

(54) YEAST SURFACE TWO-HYBRID SYSTEM FOR QUANTITATIVE DETECTION OF PROTEIN-PROTEIN INTERACTIONS

(75) Inventors: Moonsoo M. Jin, Ithaca, NY (US); Xuebo Hu, Ithaca, NY (US)

Correspondence Address: SCULLY SCOTT MURPHY & PRESSER, PC, 400 GARDEN CITY PLAZA, SUITE 300, GARDEN CITY, NY, 11530, US

(73) Assignee: Cornell University, Ithaca, NY (US)

(21) Appl. No.: 12/558,112

(22) Filed: Sep. 11, 2009

Related U.S. Application Data
(60) Provisional application No. 61/096,552, filed on Sep. 12, 2008.

(51) Int. Cl. C12Q 1/68; C12N 1/19; C40B 40/00; C40B 30/04

(52) U.S. Cl. 435/6; 435/254.2; 435/254.21; 435/254.22; 435/254.23; 506/13; 506/9

(57) ABSTRACT

The present invention provides methods and related vectors and host cells for quantitative analysis of protein interactions in eukaryotic expression system. More specifically, the invention provides a yeast surface two-hybrid (YS2H) system that can express a pair of proteins, one protein (“bait”) as a fusion to a yeast cell wall protein, and the other (“prey”) in a secretory form. When two proteins interact in this system, they associate in the secretory pathway, and the prey that would otherwise be released into the media is captured on the cell surface by the bait. Expression of the bait and the prey proteins can be designed to promote a synchronized and comparable level of expression. The affinity of two interacting molecules can be quantitatively determined by two exemplary schemes: either flow cytometric detection of antibody binding to the epitope tags fused to the prey and the bait, or the readout from a protein-fragment complementation assay (“PCA”) such as complementation of split GFP fragments fused to the prey and the bait.

### CROSS REFERENCE TO RELATED APPLICATION

This application claims the benefit of priority from U.S. Provisional Application No. 61/096,552, filed on Sep. 12, 2008.

### FIELD OF THE INVENTION

This invention relates to methods and compositions for detecting protein-protein interactions. More specifically, the present invention relates to a yeast surface two-hybrid (YS2H) system that can express a pair of proteins, one protein (“bait”) as a fusion to a yeast cell wall protein, and the other (“prey”) in a secretory form, wherein the two proteins associate in the secretory pathway, and the prey that would otherwise be released into the media is captured on the cell surface by the bait.

### BACKGROUND OF THE INVENTION

Protein-protein interactions are essential to virtually every cellular process, and understanding of these interactions is of great interest to basic science as well as to the development of effective therapeutics. Existing techniques to detect and screen pairs of interacting proteins in vivo include the yeast two-hybrid system (1) and protein-fragment complementation assay (PCA) (2-6), where the association of two interacting proteins either turns on a target gene that is necessary for cell survival or leads to the reconstitution of enzymes or green fluorescence protein (GFP) or its variants. The application of protein-protein interactions that are probed with yeast two-hybrid and PCA has been focused mainly on the interactions occurring in the nucleus or cytosol. To study interactions among secretory proteins and membrane-associated proteins, a variant of the yeast two-hybrid system has been developed for detecting protein-protein interactions occurring in the secretory pathway (7, 8). However, most existing methods are designed to map connectivity information for pairwise interactions and are not suitable for measuring the affinity between two interacting proteins, comparing interaction strength of different pairs, or ranking multiple binders to the interaction “hub” according to their binding affinity.

Quantitative estimation of protein-protein interactions in vivo will require the amount of the complex to be directly measured or the level of reconstituted reporters to be directly proportional to the strength of the interactions. Prior to the present invention, there has been no effective method for quantitative detection of protein-protein interactions in vivo.

### SUMMARY OF THE INVENTION

The present invention is directed to a yeast surface two-hybrid (YS2H) system that permits quantitative analysis of protein interactions in eukaryotic expression system. More specifically, the invention provides yeast cells and related methodology wherein a pair of proteins are expressed in the yeast cells, one protein (“bait”) as a fusion to a yeast cell wall protein, and the other (“prey”) in a secretory form. When two proteins interact in this system, they associate in the secretory pathway, and the prey that would otherwise be released into the media is captured on the cell surface by the bait. The association of the bait and the prey can be detected and quantified based on a number of schemes further described herein below.

The YS2H system provided by the present invention is particularly useful for evaluating specific interactions between antigen and antibody, identifying critical sites of allosteric activation in proteins, and isolation of candidate polypeptides that bind to a target protein.

While the YS2H system has been specifically exemplified herein using Saccharomyces cerevisiae, other yeast cells can be employed, which include, e.g., Schizosaccharomyces pombe, Pichia pastoris, and species of the Candida genus.

In one aspect, the present invention provides a yeast cell transformed with two expression cassettes, wherein the first expression cassette comprises from 5′ to 3′: a first promoter, a nucleotide sequence coding for a fusion protein between a bait protein and a yeast cell wall anchor protein wherein the fusion protein comprises a first signal sequence, and a first 3′ untranslated region; the second expression cassette comprises from 5′ to 3′: a second promoter, a nucleotide sequence coding for a prey protein in fusion to a second signal sequence, and a second 3′ untranslated region; and wherein upon expression of the bait and the prey protein in the yeast cell, the bait protein becomes anchored in the cell wall, and the prey protein is produced as a secretory protein.

The two cassettes can be provided in the same or different vectors. The yeast cell is engineered such that the bait and the prey proteins are expressed in a synchronized manner at comparable levels.

In certain specific embodiments, the first and second promoters are identical inducible promoters. In other embodiments, the first and second promoters are not identical; for example, the two promoters are the GAL1 promoter and the GAL10 promoter, respectively, of S. cerevisiae, which have been shown to provide comparable levels of expression.

In a preferred embodiment, the cell wall anchor protein is selected from the Aga2 protein or the flocculation domain of the flocculation protein (Flo1p).

In another preferred embodiment, the first and second signal sequences are independently selected from the signal sequences of the Aga2 protein, α-1 mating factor, PHA (phytohemagglutinin), and the flocculation protein (Flo1p).

While detection of the association between the bait and the prey can be achieved by a variety of means, in a preferred embodiment, detection and quantification of the association are facilitated by linking the bait protein to a first reporter molecule, and the prey protein to a second reporter molecule.

In one embodiment, the first and second reporter molecules are different epitope tags, for example, an epitope tag selected from the group consisting of Myc, FLAG, HA, 6xHis, and T7 tag.

In another embodiment, the first and second reporter molecules are complementation fragments of a protein which, upon binding to each other, reconstitute the protein. Examples of such protein include, e.g., green fluorescent protein (GFP), β-lactamase, and dihydrofolate reductase (DHFR).

In another aspect, the present invention provides a library of yeast cells, each cell in the library containing two expression cassettes, wherein the first expression cassette comprises from 5′ to 3′: a first promoter, a nucleotide sequence coding for a fusion protein between a bait protein and a yeast cell wall anchor protein wherein the fusion protein comprises a first signal sequence, and a first 3′ untranslated region; the second expression cassette comprises from 5′ to 3′: a second promoter, a nucleotide sequence coding for a prey protein in fusion to a second signal sequence, and a second 3′ untranslated region; and wherein upon expression of the bait and the prey protein in the yeast cell, the bait protein becomes anchored in the cell wall, and the prey protein is produced as a secretory protein; and wherein (1) the bait protein is the same for all of the cells in the library, and the prey proteins are different for all or at least some of the cells in the library; (2) the prey protein is the same for all of the cells in the library, and the bait proteins are different for all or at least some of the cells in the library; or (3) the bait and the prey proteins are each different for all or at least some of the cells in the library.

In still another aspect, the invention is directed to a yeast surface two hybrid (YS2H) system useful for evaluating interactions between protein pairs. The system is composed of (1) a yeast cell, (2) a vector containing two expression cassettes, wherein the first expression cassette comprises from 5′ to 3′: a first promoter, a nucleotide sequence coding for a fusion protein between a bait protein and a yeast cell wall anchor protein wherein the fusion protein comprises a first signal sequence, and a first 3′ untranslated region; the second expression cassette comprises from 5′ to 3′: a second promoter, a nucleotide sequence coding for a prey protein in fusion to a second signal sequence, and a second 3′ untranslated region; and wherein upon expression of the bait and the prey protein in the yeast cell, the bait protein becomes anchored in the cell wall, and the prey protein is produced as a secretory protein; and (3) reagents or instructions for detecting signals generated upon association of the bait and the prey proteins on the cell surface of the yeast cell.

In a further aspect, the invention is directed to a method for assessing the interactions between two proteins. The method is achieved by (1) providing a yeast cell, (2) providing two expression cassettes, wherein the first expression cassette comprises from 5′ to 3′: a first promoter, a nucleotide sequence coding for a fusion protein between a bait protein and a yeast cell wall anchor protein wherein the fusion protein comprises a first signal sequence, and a first 3′ untranslated region; the second expression cassette comprises from 5′ to 3′: a second promoter, a nucleotide sequence coding for a prey protein in fusion to a second signal sequence, and a second 3′ untranslated region; and wherein upon expression of the bait and the prey protein in the yeast cell, the bait protein becomes anchored in the cell wall, and the prey protein is produced as a secretory protein; (3) introducing the two expression cassettes into the yeast cell by transformation; (4) expressing the two proteins in the transformed yeast cell to allow association between the two proteins; and (5) detecting signals generated upon association of the bait and the prey proteins on the cell surface of the yeast cell as a basis for assessing the interactions between the two proteins.

In a specific embodiment, the assessment of the interactions between the two proteins includes determining the binding affinity between the two proteins.

In another aspect, the present invention provides a method of identifying a candidate protein which binds to a target protein from a plurality of candidate proteins. This method includes the steps of (1) providing a library of yeast cells, wherein each cell in the library comprises two expression cassettes, wherein the first expression cassette comprises from 5′ to 3′: a first promoter, a nucleotide sequence coding for a fusion protein between said target protein as a bait protein and a yeast cell wall anchor protein wherein said fusion protein comprises a first signal sequence, and a first 3′ untranslated region; the second expression cassette comprises from 5′ to 3′: a second promoter, a nucleotide sequence coding for a candidate protein as a prey protein in fusion to a second signal sequence, and a second 3′ untranslated region; wherein upon expression of the bait and the prey protein in the yeast cell, the bait protein becomes anchored in the cell wall, and the prey protein is produced as a secretory protein; and wherein the first expression cassettes are identical among all of the cells in the library, and wherein the second expression cassettes are identical among the cells except for the candidate proteins such that a plurality of candidate proteins are represented by the library; (2) expressing the proteins to allow association between candidate proteins to the target protein; (3) detecting signals generated upon association of candidate proteins to the target protein as a basis for assessing the binding interactions between a candidate protein to the target protein; (4) sorting cells in said library based on binding affinities of candidate proteins to the target protein; and (5) identifying a cell expressing a candidate protein having a selected binding affinity to the target protein.

Similarly, the present invention provides a method of identifying a candidate protein which binds to a target protein from a plurality of candidate proteins, wherein the target protein is expressed as the prey, and the candidate proteins are expressed as the bait proteins.

In a further aspect, the present invention provides a method of identifying interacting protein pairs (i.e., selection of library against library). The method comprises the steps of (1) providing a library of yeast cells, wherein each cell in the library comprises two expression cassettes, wherein the first expression cassette comprises from 5′ to 3′: a first promoter, a nucleotide sequence coding for a fusion protein between a candidate protein as a bait protein and a yeast cell wall anchor protein wherein said fusion protein comprises a first signal sequence, and a first 3′ untranslated region; the second expression cassette comprises from 5′ to 3′: a second promoter, a nucleotide sequence coding for a candidate binding partner protein as a prey protein in fusion to a second signal sequence, and a second 3′ untranslated region; wherein upon expression of the bait and the prey protein in a yeast cell, the bait protein becomes anchored in the cell wall, and the prey protein is produced as a secretory protein; and wherein the first expression cassettes are identical among the cells except for the candidate proteins such that a plurality of candidate proteins are represented by the library, the second expression cassettes are identical among the cells except for the candidate binding partner proteins such that a plurality of candidate binding partner proteins are represented by the library, and preferably, for a given candidate protein, a plurality of different candidate binding partner proteins are represented by cells in the library, and for a given candidate binding partner protein, a plurality of different candidate proteins are represented by cells in the library; (2) expressing the proteins in the yeast cells to allow association between candidate proteins to candidate binding partner proteins; (3) detecting signals generated upon association of candidate proteins to candidate binding partner proteins; and (4) sorting cells in the library based on binding affinities of candidate proteins to candidate binding partner proteins; and (5) identifying a cell expressing a candidate protein and a candidate binding partner protein having a selected binding affinity towards each other.

### BRIEF DESCRIPTION OF THE DRAWINGS

FIGS. 1a-1d. Design of the YS2H system. a, a map of the YS2H vector is drawn with restriction enzyme sites and genes labeled. The bait protein is expressed as a fusion to Aga2 on cell surface, whereas the prey protein is expressed as a secretory form. b and c, schematic diagrams of the expression cassette and protein-protein interactions (acid base coiled coils) via the secretory pathway are depicted. The prey bound to the bait is detected by antibody binding to the Myc tag (b) or by direct GFP readout from split GFP complementation (c). FLAG (DYKDDDDK) (SEQ ID NO: 1) and Myc (EQKLISEEDL) (SEQ ID NO: 2) epitope tags are fused to the C-terminal of the bait and prey proteins, respectively, and are used to measure the surface expression of the bait and the amount of the prey that is bound to the bait. d, the deletion of signal sequence for the prey and bait proteins leads to their expression in the cytosol.

FIGS. 2a-2d. Detection of coiled coil interactions by epitope expression and GFP complementation. (a) A schematic diagram (adapted from the FIG. 1 by De Crescenzo et al (17)) of the acid (En)-base (Kn) coiled coils, with ‘n’ indicating the number of heptad repeats, and the amino acid sequences of “E” and “K” being set forth in SEQ ID NO: 6 and SEQ ID NO: 7, respectively. The detection of coiled coil interactions by antibody binding to Myc tag (b) or direct GFP readout (c) using flow cytometry. Antibody binding to the Flag tag measures the level of the base coil expression on cell surface. (d) Shown are the plots of GFP complementation due to the coiled coil interactions occurring inside the cells. Numbers in each plot (b-d) indicate mean fluorescence intensity (MFI) of entire population shown in filled histogram. Thin lines represent the histograms of uninduced clones. The pairs of bait and prey are denoted for each column as bait:prey. The labels ‘K3’ and ‘E3’ indicate that the other coil is deleted from the expression vector.

FIGS. 3a-3d. The correlation of the affinity measured by SPR (17) with the detection by epitope tag or direct readout of GFP complementation due to coiled coil interactions occurring in the secretory pathway (a-b) or in the cytosol (c). The data are from three independent experiments involving different clones (mean±standard error). The smooth solid lines are drawn by connecting data points. (d) The MFI of eGFP complementation from the coiled coil interactions is plotted as a function of their on-rate, measured by SPR (17). The solid line represents a least square fit to the data points.

FIGS. 4a-4c. Detection of specific interactions between antibodies and antigens in YS2H. (a) Schematic diagram of the expression cassette used to study antigen (bait) and antibody (prey) interactions. Shown are the histograms of the interactions of the wild-type and the high affinity (HA) I domains as baits and activation-insensitive antibody, TS1/22 (b) activation-specific antibody, AL-57 (c) as preys. Filled histograms are of antibody binding to Myc and Flag tags to the induced clones. Thin black lines represent antibody binding to uninduced clones as controls. Numbers in each plot indicate mean±standard error of the MFI of the filled histograms from three independent measurements.

FIGS. 5a-5f. Discovery of allosteric activation in the I domain. (a) Cartoon diagrams of low (inactive) and high affinity (active) conformations of the LFA-1 I domains. The regions that are structurally conserved between two states are colored in grey. The regions that differ structurally are colored in magenta and yellow for the inactive and the active conformations, respectively. The metal ions in the MIDAS are shown as spheres. N- and C-termini, and α7-helix are labeled. (b) The structure of the I domain is shown in complex with the first domain of ICAM-1 (D1). Grey spheres with a white center display the positions for the hot spots for allosteric activation found in our previous study (10). The metal ion and three oxygen atoms of water molecules are depicted as spheres. The residues that coordinate to the metal ion are shown in stick models. The structures of the I domains and the complex of I domain with the ICAM-1 were modeled based on the crystal structures, as described previously (31). (c) Myc expression of the I domain library before sort and after first and second sort are shown. The numbers indicate the percentage of the clones within the gated region. Antibody binding was measured with 10 mM MgCl₂ or no metal ions with 10 mM EDTA. (d) Two activating mutations from the second sort were of F265S and L295P. Numbers in each plot indicate mean±standard error of the MFI of the filled histograms from three independent measurements. (e-f) SPR measurements of L295P (e) and F265S (f) binding to scFv AL-57. I domains were injected over the scFv AL-57 coated chip as a series of 2-fold dilutions beginning at 500 nM.

FIGS. 6a-6e. Detection of VHH binding to BoNT LC protease. (a) Specific binding of the VHHs against A-LC and B-LC was confirmed in YS2H by Myc expression. (b) New VHHs against B-LC protease were isolated by YS2H. Numbers in each plot indicate mean±standard error of the MFI of the filled histograms from three independent measurements. (c-d) SPR measurements of B8 (c) and G6 (d) binding to BoNT/A-LC. A-LC was injected at a series of 2-fold dilutions beginning at 160 nM to the B8- and 400 nM to the G6-coated chip. (e) Cysteines are highlighted in yellow box for the pair that forms a conserved disulfide bond or in orange underlined that forms extra disulfide bonds. The framework region (FR) and complementarity determining regions (CDR) are noted. The amino acid sequences of the polypeptides are set forth in SEQ ID NOS: 47-54, respectively.

### DETAILED DESCRIPTION OF THE INVENTION

The present invention provides methods and related vectors and host cells for quantitative analysis of protein interactions in eukaryotic expression system. More specifically, the invention provides a yeast surface two-hybrid (YS2H) system that can express a pair of proteins, one protein (“bait”) as a fusion to a yeast cell wall protein, and the other (“prey”) in a secretory form. When two proteins interact in this system, they associate in the secretory pathway, and the prey that would otherwise be released into the media is captured on the cell surface by the bait. Expression of the bait and the prey proteins can be designed to promote a synchronized and comparable level of expression. The affinity of two interacting molecules can be quantitatively determined by two exemplary schemes described herein: flow cytometric detection of antibody binding to the epitope tags fused to the prey and the bait, and the readout from a protein-fragment complementation assay (“PCA”) such as complementation of split GFP fragments fused to the prey and the bait.

Without being bound to any particular theory, it is believed that the quantitative nature of the YS2H system of the present invention is a result of protein-protein interactions occurring via a secretory pathway (as opposed to in the cytosol or extracellular media), and the amount of the prey protein in complex with the bait is determined by the equilibrium affinity between the two. It has been demonstrated herein that the quantitative nature of YS2H permits estimation of the affinity between two interacting proteins, particularly affinities in the range of 100 pM to 10 μM. This feature is especially useful for comparing interaction strength of different protein pairs, and ranking multiple binders to the interaction “hub” based on binding affinity. The instant YS2H system also allows examination of specific interactions between antigen and antibody, more efficient identification of critical sites of allosteric activation in proteins, and isolation of antibodies of varying affinities against target antigens, including antibodies not isolated by prior methodologies. With the incorporation of the PCA technique into the YS2H, the YS2H system can also be used to measure and compare the kinetics of protein-protein interactions.

The various features of the present invention are further described as follows.

Definitions

“Expression cassette”—The term as used herein refers to a nucleic acid molecule comprised of three basic components: a promoter sequence, an open reading frame, and a 3′ untranslated region that, in eukaryotes, usually contains a polyadenylation site. An expression cassette can be inserted and becomes part of a vector DNA used for cloning, transformation and expression.

“Open reading frame” or “ORF”—The term refers to the portion of a gene (naturally occurring or synthetic or recombinant gene) that begins with the start codon and ends with a stop codon and that encodes a protein. An ORF may include one or more intron sequences.

“Vector”—The term as used herein refers to a nucleic acid molecule that acts as a carrier of another nucleic acid molecule, wherein such other nucleic acid molecule is linked or inserted into the vector for purposes of replication and transformation.

“Prey” and “bait” proteins—The term refers to a pair of interacting proteins capable of binding to each other. As used herein specifically, a “bait” protein refers to a protein genetically engineered as fused in frame to an anchor protein or polypeptide sequence that directs the bait protein through the secretory pathway to become anchored at the cell wall location of a yeast cell. A “prey” protein is a protein engineered as linked to a signal sequence that directs the prey protein through the secretory pathway of a yeast cell in a soluble form.

“Signal sequence” or “signal peptide”—The term refers to a short (3-60 amino acids long) peptide that directs the post-translational transport of a protein (which are synthesized in the cytosol) to certain organelles such as the nucleus, mitochondrial matrix, and endoplasmic reticulum, for example. For proteins having an ER signal peptide, the signal peptides are typically cleaved from the precursor form by signal peptidase after the proteins are transported to the ER, and the resulting proteins move along the secretory pathway to their intracellular (e.g., the Golgi apparatus, cell membrane or cell wall) or extracellular locations.

“Mature protein”—For some proteins, cleavage of the signal peptide results in the mature form (i.e., the final, biologically active form) of the protein, while for other proteins, additional proteolytic processing may be required in order to generate the mature form of the protein.

“Epitope”—The term refers to the part of a macromolecule (such as but not limited to proteins) that is recognized by the immune system, specifically by antibodies, B cells, or T cells. Epitopes can be three-dimensional surface features of an antigen molecule, or linear amino acid sequences (the primary structure) rather than the 3D shape (tertiary structure) of a protein.

“Epitope tag”—The term refers to an amino acid sequence that constitutes an epitope specifically recognized by an antibody and that is fused to a heterologous protein.

“Single chain antibody” or “ScFv”—The term refers to a fusion of portions of the heavy and light chains of an antibody that retains a single active antigen-binding site.

“Protein-fragments Complementation Assay” or “PCA”—The term refers to a method whereby protein interactions are coupled to refolding of reporter proteins from cognate fragments where reconstitution of reporter enzymatic or other activity acts as the detector of a protein interaction. Specifically, the gene for a reporter enzyme is rationally dissected into two fragments. Fusion proteins are constructed with two test proteins that are thought to bind to each other, fused to the two fragments of the reporter protein, respectively. Binding of the two test proteins to each other results in the folding of the reporter protein from its fragments, which is detected by reconstitution of the enzymatic activity or other detectable characteristics of the reporter protein. Examples of PCA reporters include, e.g., green fluorescence protein (GFP), β-lactamase, and dihydrofolate reductase (DHFR).

The Yeast Surface Two Hybrid System (YS2H)—Components and Operation

In accordance with the present invention, the YS2H system is capable of expressing a pair of proteins in a synchronized manner at comparable levels in a yeast cell, with one protein (“bait”) as a fusion to a yeast cell wall protein, and the other (“prey”) in a secretory form. The bait and the prey proteins are each linked to a reporter molecule, and the interaction between of the bait and the prey can be detected and quantitated based on detecting the reporter molecules.

To achieve synchronized and comparable levels of expression of bait and prey proteins, nucleic acid sequences coding for the bait and the prey are placed within separate expression cassettes and in operable linkages to promoters and 3′ untranslated/regulatory regions in such a manner to achieve synchronized and comparable levels of expression in a yeast cell. By “comparable levels of expression” in referring to two promoters, it is meant that the two promoters have substantially the same level of transcriptional activity resulting in substantially the same amount of transcripts. By “substantially the same” it is meant the difference is not more than 30%, 25%, 20%, 15%, 10% or even 5%. For example, two identical promoters, preferably inducible promoters functional in yeast cells (e.g., the AOX1 promoter, and the FLD promoter specifically for methylotrophic yeast) can be used. Alternatively, a bi-directional promoter (such as the GAL1-GAL10 promoter (SEQ ID NO: 24) (15)) can be used to direct expression of two test proteins.

To direct the bait protein to the cell wall location, the coding sequence for the bait protein is linked in frame to a coding sequence for a signal peptide, and also to a coding sequence for a protein or polypeptide (“anchor protein or polypeptide”). The signal sequence and the anchor protein together direct the bait through the secretory pathway to become anchored at the cell wall. For example, the bait protein can be fused to the full-length yeast Aga2 protein (SEQ ID NO: 19), or the flocculation domain encoded by the FLO1 gene (32). The full-length forms of these proteins include both a signal peptide and the mature anchoring form. Alternatively, a signal peptide from a different protein can be employed, which is fused to the mature form of a cell wall protein and the bait protein.

To direct the prey protein through the secretory pathway via ER in a soluble form, the coding sequence for the prey protein is linked in frame to a coding sequence for a signal sequence that directs the prey protein through the secretory pathway in a soluble form. Examples of suitable signal sequences include those of yeast secretory proteins or cell wall proteins, e.g., the signal sequence of the yeast α-1 mating type factor (SEQ ID NO: 16), the signal peptide of the Aga2 protein (SEQ ID NO: 17), PHA (phytohemagglutinin)(33), and Flo1p (flocculation protein), for example. The signal peptide for the prey should be chosen to provide comparable (i.e., substantially the same) level of secretion as that of the bait. For example, the signal peptide for the prey can be the same as that of (i.e. within) the anchor protein for the bait. Generally speaking, proteins selected to the prey are soluble proteins in nature and do not contain motifs that dictate the localization of the prey protein in a particular intracellular location.

The binding interactions between the bait and the prey can be detected by various means. In one embodiment, the binding interaction is detected by using an antibody that specifically recognizes the prey protein independent of its binding to the bait. Antibody binding to the prey in complex with the bait can then be determined by various means, including e.g., ELISA.

In another embodiment, the bait and the prey are separately linked to reporter molecules in order to detect the binding interaction between the bait and the prey. In accordance with one embodiment of the present invention, a reporter molecule can be an epitope tag which, upon binding by a specific antibody, generates a detectable signal. Suitable epitope tags for attachment to a bait or a prey include FLAG (DYKDDDDK) (SEQ ID NO: 1), Myc (EQKLISEEDL) (SEQ ID NO: 2), HA (YPYDVPDYA, SEQ ID NO: 3), 6Xhis (HHHHHH, SEQ ID NO: 4), and T7 tag (MASMTGGQQMG, SEQ ID NO: 5), among others. Antibody binding to epitope tags can be detected and quantitated by a variety of means well-known in the art, including e.g., flow cytometry.

In another embodiment, the reporter molecule linked to the bait and the reporter molecule linked to the prey are two fragments of a protein which, upon binding to each other, reconstitute the protein that is detectable based on the enzymatic activity or other characteristics (e.g., fluorescence) of the reconstituted protein—the so-called protein-fragments complementation assay (PCA) described above. Examples of PCA reporters include, e.g., green fluorescence protein (GFP), β-lactamase, and dihydrofolate reductase (DHFR). As illustrated herein below, NeGFP (containing residues Val-2 to Ala-155 of eGFP) (SEQ ID NO: 21) and CeGFP (Asp-156 to Lys-239 of eGFP) (SEQ ID NO: 23) can be attached to a bait and a prey, respectively, to successfully reconstitute eGFP protein upon binding of the bait and the prey with each other. In preferred embodiments, PCA reporters such as eGFP are employed to link to small size bait and prey proteins, e.g., proteins of a Mw of less than 10 kD, or proteins of fewer than 100 amino acids, preferably fewer than 85, 75, 50, 40, 30, 25, 20, or even 15 amino acids.

The expression cassette for expression of the bait protein and the expression cassette for expression of the prey protein can be placed on a single vector, or on separate identical or different vectors. In a preferred embodiment, both bait and prey expression cassettes are placed within a single vector. The vector or vectors carrying the bait and prey expression cassettes can contain additional nucleotide sequence elements as appropriate, including, e.g., an origin of replication, and a selectable marker gene suitable for identification of cells containing the vector.

The vectors are introduced into yeast cells by transformation for expression of the bait and the prey proteins. The vectors can be integration vectors (i.e., capable of mediating integration of the vector sequence including the expression cassettes into the yeast chromosome), or episomal vectors such as plasmids. Yeast cells suitable for use in the practice of the present invention include cells of Saccharomyces cerevisiae, Schizosaccharomyces pombe, species of the Candida genus, and species of methylotrophic yeast such as Pichia pastoris. A preferred yeast species is S. cerevisiae.

Transformants containing both the bait and prey vectors (if the two expression cassettes are on different vectors) or containing the single vector that carries both expression cassettes can be identified and selected based on selectable markers on the vectors. Upon expression of the bait and the prey proteins in the yeast cell (e.g., by activating an inducible promoter), the two proteins interact and associate in the secretory pathway. The bait protein ultimately becomes displayed on the cell surface by way of being anchored to a cell wall protein, and the prey that would otherwise be released into the media is captured on the cell surface by the bait.

The binding interaction between the bait and the prey can be detected by assaying the signals generated through the reporter molecules attached to the bait and the prey.

When the bait and the prey are each linked to a different epitope tag, the binding affinity of the two proteins can also be quantitatively assessed, based on the Langmuir binding isotherm model. In this case, the Langmuir equation is then given by

[bait:prey]/[bait]=[prey]/([prey]+K_{D}) (1)

where [bait:prey], [bait], and [prey] denote the concentrations of the bait in complex with the prey, the bait in total, and the prey in total, respectively. As illustration, assuming the bait is attached with a FLAG tag and the prey with a Myc tag, by replacing [bait:prey] and [bait] with antibody binding to Myc (MFI_Myc) and FLAG tag (MFI_FLAG), respectively, and taking into consideration of the MFI (mean fluorescence intensity) ratio (α) of anti-Myc to anti-FLAG antibody binding to equal copies of Myc and FLAG tags, the Langmuir equation rearranges into

1/MFI_Myc=α⁻¹(1+K_{D}/[prey])/MFI_FLAG (2)

With a measured prey concentration in the media, one can readily deduce the binding dissociation constant K_{D}.

As illustrated hereinbelow, this quantitative assessment in the instant YS2H system is applicable to binding interactions with an estimated K_{D} in the range of approximately 100 pm −10 μM. As shown below, the instant YS2H system capable of detecting interactions of an affinity within this range has effectively permitted examination of specific interactions between antigen and antibody, identification of hot spots of allosteric activation in integrins, and isolation of camelid heavy chain only antibodies against botulinum neurotoxin.

When the YS2H employs a PCA reporting scheme, the MFI of reconstituted GFP has been found to have a linear correlation with the rate at which the two proteins associate (on-rate) to initiate split GFP assembly. Therefore, the binding kinetics of two test proteins can be assessed, and kinetics of different interacting protein pairs can be compared.

Appropriate expression vectors, untransformed host cells, detection reagents and other useful materials can be packaged into a kit or container as appropriate for practicing the invention based on the instant YS2H system.

Applications of the YS2H System

It has been demonstrated herein that the quantitative nature of YS2H permits estimation of the affinity between two interacting proteins, particularly in the range of 100 pM to 10 μM. This feature is especially useful for comparing interaction strength of different protein pairs, and ranking multiple binders to the interaction “hub” based on binding affinity.

Further, the instant YS2H system allows identification of critical sites of allosteric activation in proteins. For example, a yeast library can be constructed, with each cell of the library expressing an antibody anchored in the cell wall (bait) recognizing a protein in its activation conformation and a candidate mutant variant of the protein (prey) generated from e.g., error-prone PCR products of the wild type protein. The library can be screened and sorted with an antibody specific for an epitope tag on the bait using a magnetic affinity cell sorter. With successive sorting, there should be a gradual increase in the percentage of the population of cells that showed the desirable signal above the background level. The sorted population of cells can be plated to identify the candidate mutant clone having an activation conformation. As illustrated in the Examples herein below, the use of YS2H has allowed an efficient identification of two activating mutations in the LFA-I domain which were previously found based on prior methodologies only after tedious screening of a large number of yeast cells.

The instant YS2H system also allows for the examination of specific interactions between antigen and antibody, and isolation from a library of antibodies having varying affinities against target antigens. In effect, the use of instant YS2H to screen for antibodies of desired affinities serves as a microbial analog of the mammalian immune system's antibody affinity maturation. “Affinity maturation” is a process that occurs in a mammal wherein cycles of mutation and evolutionary selection produce antibodies that bind their targets with higher affinity. Using the instant YS2H, a library of mutant antibody candidates are displayed on the surface of yeast cells via fusion with cell wall proteins, and the target antigen is expressed as the prey. Yeast cells are then screened based on affinity sorting (e.g., magnetic affinity cell sorting or flow cytometry sorting). Antibodies with desirable affinities towards the target antigen can be selected.

In a further aspect, the YS2H system can also be used to measure and compare the kinetics of protein-protein interactions with the incorporation of the PCA technique into the YS2H.

### EXAMPLE-1

### Experimental Procedures

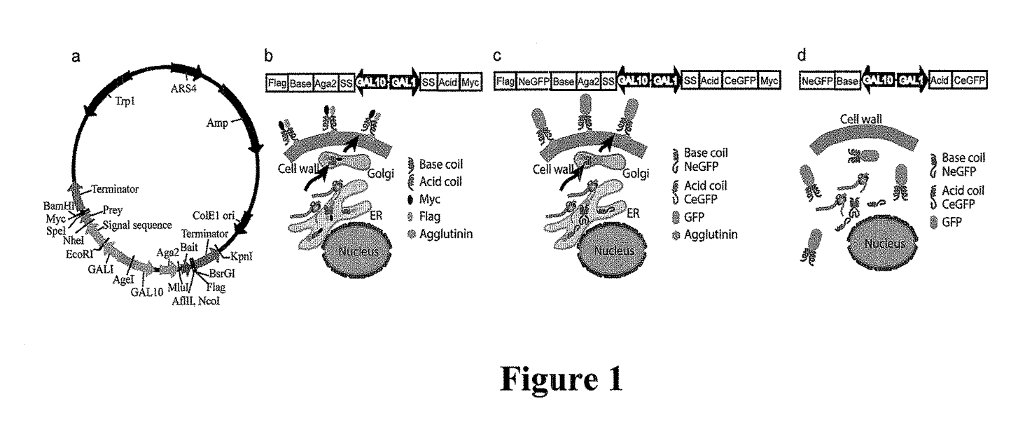

YS2H Vector Design—Plasmid pCTCON was used as a backbone for constructing the YS2H vector (see FIG. 1a). A PCR fragment containing GAL10 promoter (SEQ ID NO: 26), the AGA2 coding sequence (SEQ ID NO: 18), eGFP gene, FLAG tag, and terminator was inserted into the pCTCON by AgeI/KpnI sites. To express prey proteins as secretory forms, the AGA2 sequence under the GAL1 promoter (SEQ ID NO: 25) was removed by replacing an EcoRI/BamHI fragment with the fragment consisting of a signal sequence, either the signal sequence of Aga2 (SEQ ID NO: 17) or the signal sequence of α-1 mating factor (SEQ ID NO: 16), and prey. The cDNA coding for the variable domains of AL-57 was obtained from the expression plasmid (kindly provided by Dr. Shimaoka at Harvard Medical School). The variable domains of TS1/22 were cloned from the hybridoma (ATCC). VH and VL cDNAs were connected with four repeats of a Gly-Gly-Gly-Gly-Ser (SEQ ID NO: 27) linker sequence to produce scFv.

Yeast Transformation, Magnetic Affinity Cell Sorting, and Library Construction—The plasmid encoding a specific pair of prey and bait proteins was introduced into yeast cells using a commercial reagent (Frozen-EZ Yeast Transformation II Kit, Zymo Research). Transformed yeast cells were grown in a solid medium plate for 48 h. A mutagenesis library of LFA-1 I domain was constructed by electroporation of a mixture of a MluI/NcoI linearized vector and error-prone PCR products of the 1 domain (Asn-129 to Thr-318) into yeast, as described previously (10). After transformation, the yeast libraries were grown in selective dextrose liquid medium at 30° C. with shaking for 24 h and induced in selective galactose media for 24-48 h at room temperature with shaking. To construct the variable domain of heavy chain from heavy chain-only antibody (VHH) yeast library, cDNA encoding VHH library was amplified by PCR using the primers shown in Table 2, which were designed based on the primers used by Maass et al. (11). VHH cDNA PCR product was first ligated into the YS2H vector using NheI/BamHI sites and then was transformed into XL1-Blue (Stratagene) by electroporation. The plasmids extracted from about 5×10⁶ colonies were transformed into EBY100 by a lithium acetate method (12). A single colony of EBY100 from fresh plate was inoculated into 10 ml of YPDA medium and cultured at 30° C. with shaking at 225 rpm for 16 h. The cells were then inoculated into 100 ml of YPDA at 0.5 A₆₀₀ and cultured for another 4 h until A₆₀₀ reaches 2. The cells were washed twice in water and resuspended in 10.8 ml of transformation mix buffer (7.2 ml of 50% polyethylene glycol, 1.1 ml of 1 M LiAc, 1.5 ml of 2 mg/ml single strand carrier DNA, and 150 μg of library plasmid in 1.0 ml water). The mixture was then incubated at 42° C. for 50 min. After incubation, the cells were cultured into 100 ml of selective dextrose liquid medium for 24 h and induced in selective galactose medium for 24-48 h. Library construction by homologous recombination or the lithium acetate method produced a library size of 10⁶-10⁷. The libraries of LFA-1 I domain and VHH were sorted with anti-Myc antibody using magnetic affinity cell sorting as described previously (10).

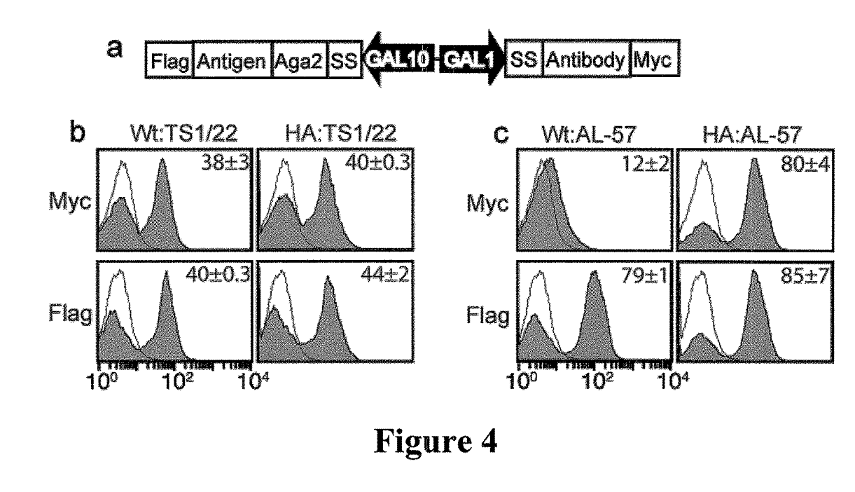

Immunofluorescence Flow Cytometry—Antibodies used in this study were the anti-c-Myc antibody 9E10 (ATCC), anti-FLAG, and phycoerythrin-labeled goat polyclonal anti-murine antibodies (Santa Cruz Biotechnology, SantaCruz, Calif.). To measure the surface expression of specific prey and bait proteins using flow cytometry, one to five colonies from solid medium plate were inoculated together to obtain averaged values. After induction, the cells were harvested, washed in 100 μl of the labeling buffer (phosphate-buffered saline with 0.5% bovine serum albumin), and then incubated with ligands at 10 μg/ml in 50 μl of the labeling buffer for 20 min with shaking at 30 ° C. The cells were then washed and incubated with secondary antibodies at 5 μg/ml in 50 μl of the labeling buffer for 20 min at 4° C. Finally, the cells were washed once in 100 μl and suspended in 100 μl of the labeling buffer for flow cytometry (FACScan, BD Biosciences). For detecting TS1/22 binding (see FIG. 4b), goat polyclonal anti-murine antibody was used as a primary antibody.

Protein Expression—The I domains were expressed in Escherichia coli BL21 DE3 (Invitrogen) as inclusion bodies and refolded and purified by an S75 size exclusion column connected to fast protein liquid chromatography (GE Healthcare) (10). AL-57 as a single-chain format (scFv AL-57) was expressed using the protocol for I domain production, except that 3 mM cystamine and 6 mM cysteamine were added to the refolding buffer. Full-length BoNT/A and BoNT/B-LC encoding DNA (amino acids1-448 of A-LC and1-440 of B-LC) were synthesized employing codons optimal for expression in E. coli. A-LC and B-LC containing hexahistidine tags at both termini were produced using a pET14b vector. To express VHHs insoluble forms, VHH cDNAs were inserted into the pET20b expression vector (Novagen). Soluble VHH was expressed in E. coli BL21 DE3, extracted by sonication, and purified using a nickel nitrilotriacetic acid column. Eluted VHHs were then injected into an S75 size exclusion column for further purification.

SPR Analysis—A protein-coupled or a control mock-coupled CM5 sensor chip was prepared using an amine coupling kit (BIAcore, Piscataway, N.J.), as described previously (10). SPR was measured using a Biacore (BIA2000). I domains were injected over the chip in 20 mM Tris-HCl, pH8.0, 150 mM NaCl, 10 mM MgCl₂ at a flow rate of 10 μl/min at room temperature. VHHs were injected over the chip in 20 mM Tris-HCl, pH 8.0, 150 mM NaCl at a flow rate of 10 μl/min at room temperature. The chip surface was regenerated by flowing 20 μl of 10 mM Tris-glycine, pH 1.5 buffer.

### EXAMPLE-2

### Results and Discussion

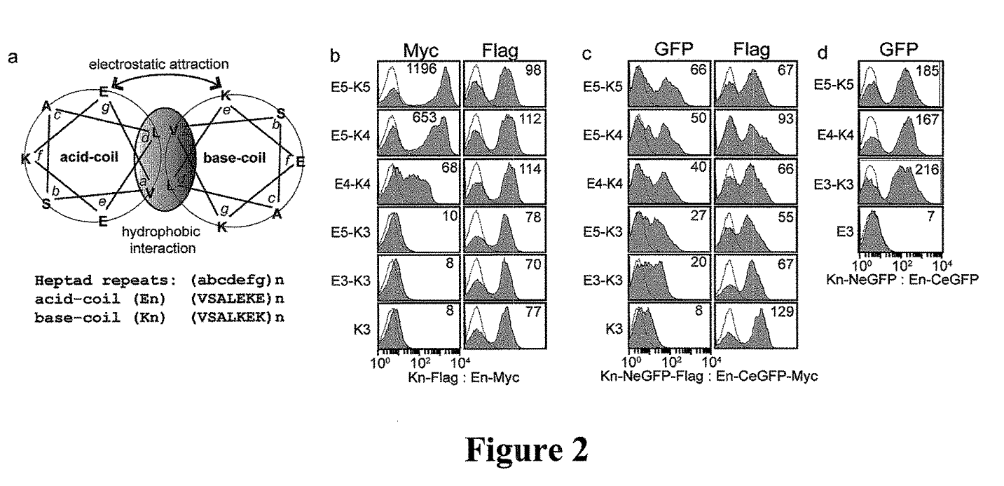

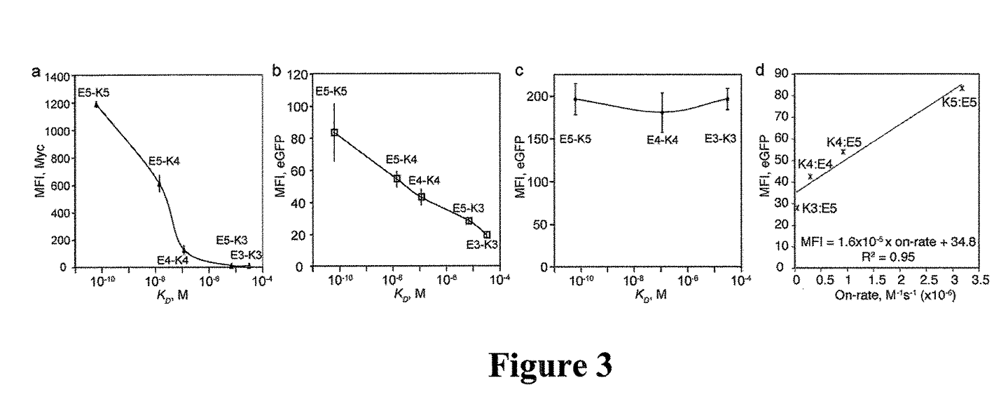

The Design of the YS2H—YS2H is built on a yeast display system (13,14), which expresses, under the control of the GAL1 promoter, a protein of interest as a fusion to Aga2. Aga2 connects to the β-glucan linked Aga1 to form a cell wall protein called agglutinin. To extend this methodology to the expression of a pair of proteins, an additional expression cassette under the GAL10 promoter was inserted into the yeast display vector, pCTCON (13, 15). Comparable expression of eGFP by GAL1 and GAL10 promoters was observed using two different plasmids that were constructed to express eGFP under either GAL1 or GAL10 promoters. The final YS2H vector (FIG. 1a) was designed to express the bait protein under the GAL10 promoter as a fusion to Aga2 and the prey protein under the GAL1 promoter without Aga2 fusion. The signal sequence used was either that of Aga2 (for the data in FIGS. 2 and 3) or the α-1 mating factor (for the data in FIGS. 4-6) (16). The expression level of prey proteins with α-1 mating factor was comparable with those containing the Aga2 signal sequence. FLAG and Myc tags were fused to the C-terminal of the bait and prey proteins, respectively, and were used to examine the surface expression of the bait and the amount of the prey that was bound to the bait (FIG. 1b). To incorporate the PCA technique into the YS2H system, the nucleic acid sequences encoding enhanced eGFP fragments (3) were inserted downstream of the bait (NeGFP containing residues Val-2 to Ala-155) (SEQ ID NO: 21) and the prey (CeGFP with Asp-156 to Lys-239) (SEQ ID NO: 23) to monitor the interaction by GFP readout (FIG. 1c). The deletion of the secretory signal sequence of the prey and bait proteins caused this pair to express in the cytosol (FIG. 1d), which can he used to compare protein-protein interactions occurring in the secretory pathway versus cytosol.

The Validation of the Yeast Surface Two-hybrid System Using Coiled Coil Interaction—To validate that antibody binding to the Myc tag or GFP readout correlates with the strength of molecular interactions in YS2H, five pairs of acid (En) and base (Kn) α-helices of varying heptad repeats (n) that associate into coiled coils were expressed (FIG. 2). These coiled coils were designed de novo to have affinities (K_{D}) in the range of 100 μM (E5/K5) to 100 μM (E3/K3) with higher affinity for longer helices through hydrophobic interactions at the interface and electrostatic attraction between the oppositely charged residues from each helix (17). Myc expression (mean fluorescence intensity (MFI), measured by antibody binding to Myc tag) exhibited a strong correlation with the interaction affinity within the range of 100 pM to 10 μM K_{D} for E5-K5 to E5-K3 (FIGS. 2b and 3a). With GFP complementation, this correlation extended beyond 10 μM K_{D}, and the difference between E5-K3 and E3-K3, corresponding to the affinity range of 10 μM to 100 μM, was clearly discernible (FIGS. 2c and 3b). The Myc expression and GFP complementation were close to the level of background when the acid coil (K3 in FIGS. 2b and 2c) was deleted, indicating a lack of spontaneous complementation of the two split GFP fragments. The level of surface expression of the bait protein measured by antibody binding to the FLAG tag was relatively invariant (FIGS. 2b and 2c), supporting the idea that the difference in the amount of the prey protein was solely due to the difference in its affinity to the bait. In contrast to a quantitative correlation between the strength of protein-protein interactions and GFP complementation, the acid and base coil interactions occurring in the cytosol (expression of the coils without secretory signal sequence) led to the complementation of split GFP that lacks correlation with the strength of coiled coil interactions (FIGS. 2d and 3c). However, GFP complementation for these pairs was still due to specific interaction between acid and base coils, evidenced by the absence of fluorescence when the base coil was deleted from NeGFP (FIG. 2d).

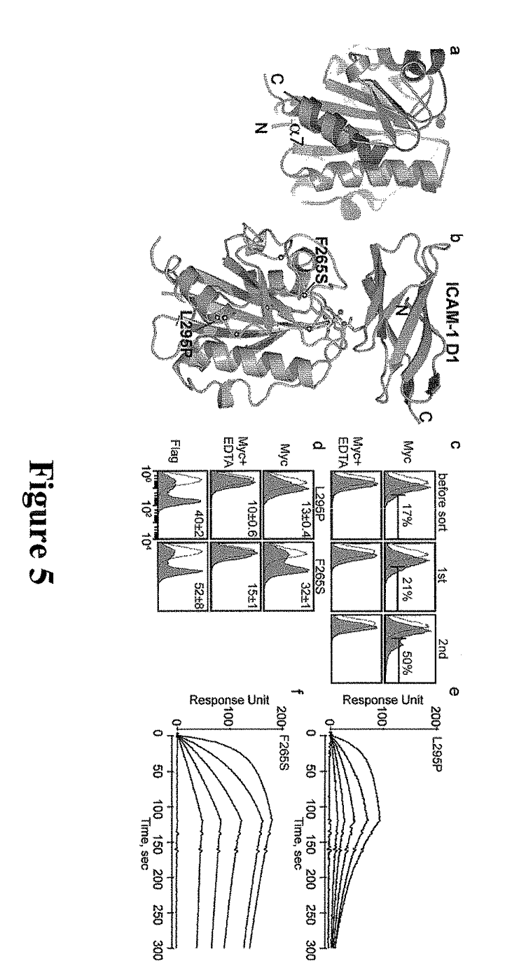

YS2H Detects Specific Interactions of Antibodies and Antigens—To investigate a potential use of YS2H for antibody discovery, it was first examined whether YS2H could detect specific interactions of known pairs of antigen and antibody. As a model system, a ligand-binding domain of the integrin LFA-1 (known as the Inserted or I domain) and monoclonal antibodies specific to LFA-1 I domain were chosen (FIG. 4). The I domain exists in two distinct conformations that correspond to low and high affinity states to its ligand, intercellular adhesion molecule-1 (ICAM-1) (FIGS. 5a and 5b). Although the I domain in isolation is predominantly in an inactive, low affinity conformation, the mutations that would favor the active conformation were found to induce high affinity binding of the I domain to the ICAM-1. For example, the mutations of K287C and K294C (high affinity or “HA” I domain) designed to stabilize by disulfide bond the position of the α7-helix into active conformation led to an increase in the affinity to ICAM-1 by 10,000-fold over the wild-type I domain (18).

The antibodies that were expressed as scFv formats included the activation-insensitive antibody (TS1/22) (19), binding to both inactive and active I domains, and the activation-dependent antibody (AL-57) (10, 20), which binds only to the active I domain. The interaction between antigen and antibody was measured by the detection of Myc tag fused to the antibody at the C terminus (FIG. 4). A tag-based assay was chosen instead of GFP complementation because it was found that the I domain fused to NeGFP did not express (no antibody binding to FLAG tag), presumably because of the quality control machinery in protein secretion (21) that prohibits misfolded proteins to be secreted. This is in contrast to the expression of split GFP with a fusion of short coils, e.g. K3-NeGFP in FIG. 2c. Therefore, it appears that when NeGFP is fused to the I domain that by itself requires proper folding for secretion, the I domain fusion to NeGFP becomes completely misfolded and does not pass the quality control for secretion.

Myc tag expression in YS2H was in agreement with the specificities of monoclonal antibody AL-57 and TS 1/22 against the LFA I domain; although the clones expressing TS1/22 displayed Myc expression either with the wild-type or with the HA I domains as antigens (FIG. 4b), the AL-57 clones exhibited Myc expression only with the HA I domain (FIG. 4c).

Discovery of Activating Mutations in the LFA-1 I Domain—Next, the ability of the YS2H system was examined in isolating activating mutations in antigens that exhibit two different activation states. With the expression of AL-57 scFv and the error-prone PCR products of the wild-type I domain in YS2H, yeast library was constructed and sorted with anti-Myc antibody using a magnetic affinity cell sorter. With successive sorting, there was a gradual increase in the percentage of the population of cells that showed Myc expression above the background level (FIG. 5c). After two rounds of sorting, the cells were plated to yield individual clones, from which four clones were sequenced and tested for Myc expression. Of the four, three contained a mutation of F265S, and one contained L295P (FIG. 5d). These two mutations belonged to a long list of activation hot spots that were identified in a previous study (10), where a large number of yeast cells were sorted and analyzed for their binding to exogenous AL-57 or ICAM-1-Fcγ.

The mutations of L295P and F265S were previously found to contribute to an increase in the binding of the I domain to ICAM-1 at 6 and 152%, respectively, of the HA I domain binding to ICAM-1 (10). To directly measure the affinity of scFvAL-57 to I domain variants, a SPR technique was used (FIGS. 5e and 5f). A first order Langmuir adsorption equation was fitted to the sensograms to obtain the kinetic and equilibrium binding constants. The equilibrium dissociation constants (K_{D})) of L295P and F265S to scFv AL-57 were 243 and 15.7 nM, respectively, in agreement with higher Myc expression with F265S in our system. AL-57 binding to the LFA-1 I domain depends on the presence of metal ions at the top of the I domain, known as the metal ion-dependent adhesion site (FIG. 5b) (22). This was also confirmed by the decrease in the Myc tag expression when EDTA was added at 10 mM to the cells during labeling (FIGS. 5c and 5d).

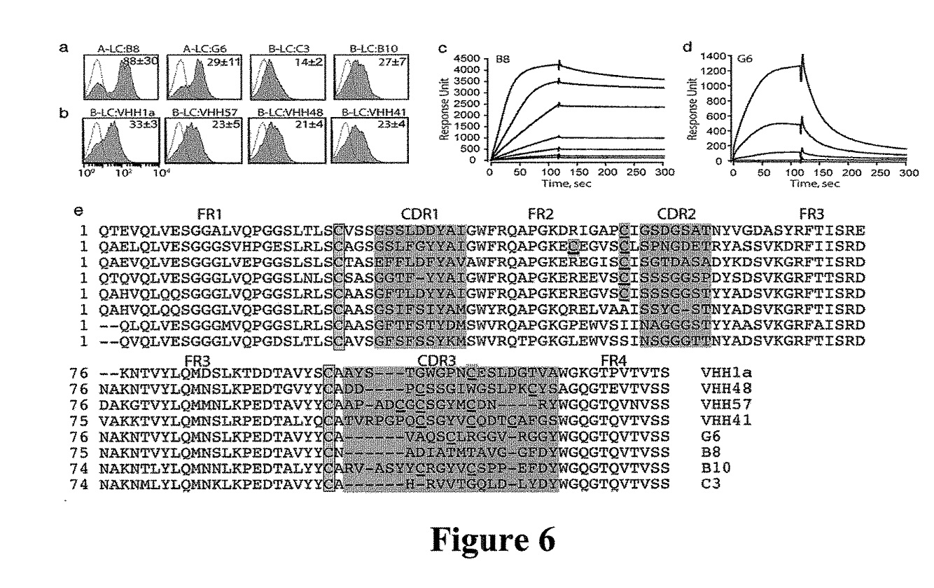

Antibody Discovery: VHH against Botulinum Neurotoxin Protease—Approximately half of the IgGs in camelid sera are heavy chain-only antibodies devoid of light chains (11, 23). Because of the lack of light chains, antigenic specificity of the heavy chain-only antibodies is limited to a variable domain of the heavy chain. VHH agents were sought that would bind and inhibit the LC protease domains of Botulinum neurotoxins (BoNTs) as components in therapeutic agents for the treatment of botulism. In prior work (Maass et al. (11), alpacas were immunized with BoNT LCs of serotype A (A-LC) followed by serotype B (B-LC). Phage display techniques were then used to identify VHHs from these alpacas with affinity for the BoNT LC proteases. Two A-LC binding VHHs (B8 and G6) and two B-LC binding VHHs (B10 and C3) were selected for testing with the YS2H system (FIG. 6a). Myc tag expression was found to be highest in the clone expressing VHH-B8 and BoNT/A-LC, whereas the binding level of the other VHHs was lower with MFI ranges from 14 to 29.

To confirm that the level of Myc expression correlates with the solution affinity of VHH to LC, a SPR technique was used (FIGS. 6c and 6d). A series of 2-fold dilutions of A-LC was injected into a chip coated with B8 and G6. The K_{D} values of B8 and G6 to BoNT/A-LC were estimated to be 2.3 and 230 nM, respectively. The 100-fold difference in the affinity was mainly clue tot he 34-fold difference in the dissociation rate (k_{off}=5.18×10⁻⁴ s⁻¹ for B8 versus 1.82×10⁻² s⁻¹ for G6), with the association rate differing by only 3-fold (kₒₙ=2.26×10⁵ M⁻¹s⁻¹ on for B8 versus 7.61×10⁴ M⁻¹s⁻¹ for G6).

To validate the use of YS2H for antibody discovery, a yeast antibody library was constructed by transforming cells with the alpaca immune cDNA library as prey and the BoNT/B-LC gene as bait. Yeast cells enriched after two rounds of magnetic affinity cell sorting with anti-Myc antibody began to show an increase in Myc tag expression. Of 30 clones that were tested individually, 14 clones displayed positive expression of Myc tag. Eleven of these 14 clones were found to be unique clones, including B10 and C3, which were originally isolated by phage display. Anti-Myc antibody binding of the newly isolated nine clones was in the range of 21-33 MFI units, which is lower than that of B8 binding to A-LC (Myc expression of four selected clones are shown in FIG. 5b). Overall, anti-B-LC VHHs isolated by both the phage display and YS2H were low affinity binders (K_{D}=100 nM to 1 μM), suggesting a lack of the high affinity binders to B-LC protease in alpaca immune library.

Sequence analysis of the VHHs identified by phage or YS2H revealed that VHHs contain two, four, or six cysteines, which would result in up to two extra disulfide bonds in addition to the one that is conserved in all immunoglobulin fold domains (FIG. 6e). Of the nine VHHs newly isolated by the YS2H system, three VHHs contain six cysteines, and the other six VHHs contain four cysteines. In contrast, either two or four cysteines were dominant in VHHs that were isolated by phage display. The VHHs identified by a phage display system may be limited to those that fold properly in a bacterial expression system. VHHs containing extra disulfides may fold improperly in bacteria, whereas the formation of correct disulfide bonds is much less problematic in yeast.

Discussion

The YS2H system of the invention has been demonstrated above to be highly efficient in the detection and discovery of protein-protein interactions. The quantitative nature of YS2H is dictated by the fact that protein-protein interactions occur via a secretory pathway, and the amount of the prey protein in complex with the bait is determined by the equilibrium affinity between the two. With the use of a PCA technique, the system can be designed to discriminate different pairs of protein-protein interactions according to their kinetics of binding.

The utility of in vivo methods for quantitative estimation of binding affinity extends to the cases where one aims to increase the affinity of weak interactions between antigen and antibody and to engineer high affinity ligands and receptors that can potentially serve as agonists or antagonists. As an example, a prokaryotic system capable of co-expression of antigens anchored on the inner membrane of bacteria and single chain variable fragments (scFv) as soluble form was efficient in affinity screening and maturation (24). Therefore, co-expression of antigen and antibody through eukaryotic secretory system will further enable screening of antibody libraries against the proteins that require eukaryotic folding machinery or that undergo post-translational modifications.

The amount of the prey bound to the bait in the instant YS2H system follows the Langmuir binding isotherm model. With the expression system used in this study, the concentration of prey proteins released into the media is far larger than that of the bait proteins, which is fused to Aga2. Under the mating conditions, the number of agglutinin goes up to 10,000 copies/cell (25), which approximates the concentration of the bait proteins to be 1.7 nM at 10⁸ cells/l ml of culture medium. The Langmuir equation is then given by [bait:prey]/[bait]=[prey]/([prey]+K_{D}) where [bait:prey], [bait], and [prey] denote the concentrations of the bait in complex with the prey, the bait, and the prey, respectively. By replacing [bait:prey] and [bait] with antibody binding to Myc (MFI_Myc) and FLAG tag (MFI_FLAG), respectively, and taking into consideration of the MFI ratio (α) of anti-Myc to anti-FLAG antibody binding to equal copies of Myc and FLAG tags, the Langmuir equation rearranges into 1/MFI_Myc=α (1+K_{D}/[prey])/MFI_FLAG. From this equation (with measured values of α=15 and [prey]=10 nM), the K_{D} values predicted for coiled coil interactions (E5-K5, E5-K4, E4-K4, and E5-K3) closely approximated the K_{D} values measured by SPR (17) (Table 1).

The quantitative nature of YS2H in measuring protein-protein interactions extends to antigen and antibody interactions. The binding affinity of scFv TS1/22 to the wild-type and HA I domain, scFv AL-57 to the HA I domain, and the F265S ranged between 148 and 237 nM K_{D}, whereas it was 440 nM for the binding of L295P to scFvAL-57. SPR measurement of the binding of I domain variants to scFv AL-57 estimated that although F265S and HA I domains have comparable affinity to scFv AL-57, L295P showed much lower affinity (FIGS. 5e and 5f, and Table 1). The predicted affinity for VHH-B8 and VHH-G6 binding to A-LC is 38 and 143 nM, compared with the measured K_{D} of 2.3 and 230 nM, respectively. Overall, the affinity predicted by the level of antibody binding to Myc and FLAG tag in the instant system agreed well with the measured affinity in the range of 1 nM to 1 μM K_{D} (Table 1).

Although antibody binding to the Myc tag for protein-protein interactions higher than 10 μM K_{D} reduced to the level of background (FIG. 3a), the detection by GFP complementation spanned a larger range of affinities, exhibiting a linear decrease in the fluorescence with an increase in K_{D} in log scale (FIG. 3b). This is attributed to the fact that reconstituted GFP does not dissociate (or complementation is irreversible), such that the complemented GFP is functional whether or not the prey and the bait exist as a complex on the cell surface. Therefore, the dominant factor that determines GFP reconstitution is the rate at which two coils associate (on-rate) to initiate split GFP assembly. Notably, when the MFI of reconstituted GFP was plotted against the measured on-rates (17) for E5-K5, E5-K4,E4-K4, and E5-K3 (on-rate is unavailable for E3-K3), a linear trend was obtained with a R² value of 0.95 (FIG. 3d). However, the use of GFP complementation was limited to the study of coiled coil interactions or other small proteins, because the I domain fused to the split GFP did not express on the surface. Therefore, to apply a PCA technique to detect diverse protein-protein interactions through the secretory pathway, split GFP needs to be optimized not to interfere with the folding of the bait and prey proteins. The additional parameter to be optimized is the length of the linker connecting split GFP to the proteins to enable GFP complementation for a wide range of size variation in proteins and topological variation between the binding interface and the GFP fusion site (6).

The fact that GFP complementation occurs by protein-protein interaction through the secretion process explains its quantitative correlation with the strength of protein-protein interactions. This is in contrast to a previous finding (4) that GFP complementation from protein interactions occurring in the cytosol only indicates the presence of the interaction, and fluorescence intensity is relatively invariant with the affinity of two proteins. Indeed, when the two coils were expressed in the cytosol with the deletion of the secretory signal sequence, it was found in the above experiment that overall fluorescent intensity was higher, and the complementation of split GFP lacked correlation with the strength of coiled coil association (FIGS. 2d and 3c). Because of the irreversible complementation of split GFP, after 24-48 h of induction, it is the concentration of two interacting proteins in the cytosol that determines the GFP complementation rather than their interaction strength.

Systems such as ribosomal (26), phage (27), and yeast (14) displays provide efficient means to couple genotype and phenotype and to screen library for protein engineering and antibody discovery. A typical screening process of antibody libraries requires exogenous antigens in their soluble form. Co-expression of two proteins within the same display system, e.g. the fusion of antigen and antibody into split phage coat protein (28) and the expression of antigen and antibody in bacterial periplasm as bait and prey proteins (24), can be particularly useful if target antigens are hard to express or unstable in solution. The YS2H system of this invention permits selection of antibodies against antigens that need to be expressed in eukaryotes. Other applications of YS2H include expression of heterodimeric proteins, as demonstrated by similar platforms for expression of heterodimeric mammalian proteins such as major histocompatibility complex II α and β subunits (29) and antibodies in Fab format (30). The use of this YS2H system to quantify and discover protein-protein interactions is not necessarily limited to the study of secretory proteins, because many proteins in nonsecretory cellular compartments or cytosol will maintain native conformations and interactions.

An in vivo tool to map protein interactions has generated a large set of protein interactions, particularly among yeast proteins (6). The readouts from the assays such as yeast two-hybrid and PCA are of cell growth caused by the expression of auxotrophic markers or reconstitution of enzymes and fluorescent proteins and are suitable for determining the presence or absence of protein interactions. In the case of protein network “hubs” in the binary protein interactome, i.e. the proteins interacting with a large number protein partners, the information on the strength of pairwise interactions may provide an important insight into the flow of biological signals orchestrated by the protein hubs. The newly developed YS2H system of this invention is well poised to implement such tasks. For example, in YS2H the hub proteins and known interacting partners are expressed as a pair of the bait and the prey, respectively, and the strength of pairwise interactions can be quantitatively estimated by antibody binding to fusion tags. Additionally, one can discover unknown interaction partners by expressing a library of hypothetical interacting partners in YS2H.

TABLE 1
Comparison of equilibrium dissociation constants (K_{D})
predicted from YS2H vs. directly measured using surface
plasmon resonance.

| | Predicted from YS2H | SPR |
|---|---|---|
| Interaction pairs | (nM; mean ± sem) | measurements (nM) |
| E3:K3 | 1269.5 ± 53 | 32000 ± 3000* |
| E5:K3 | 1292.8 ± 37 | 7000 ± 800* |
| E4:K4 | 150.7 ± 48 | 116 ± 8* |
| E5:K4 | 18.3 ± 3 | 14 ± 1* |
| E5:K5 | 0.49 ± 0.2 | 0.063 ± 0.005* |
| HA:AL57 | 150.9 ± 8 | 32.6 ± 0.28** |
| F265S:AL57 | 237.4 ± 11.6 | 15.7 ± 0.03** |
| L295P:AL57 | 439.7 ± 13.6 | 243 ± 3.9** |
| VHH-B8:A-LC | 38.2 ± 13.3 | 2.3 ± 0.08** |
| VHH-G6:A-LC | 143.0 ± 41.9 | 230 ± 1.3** |

*The values are from the paper by De Crescenza et al (17). Shown are mean ± 95% confidence interval.
**The values are measured from this study. Shown are mean ± sem, estimated from BIAevaulation software from Biacore.

TABLE 2

DNA sequences of forward (f) and reverse (r) primers are shown.

| | |
|---|---|
| Product | Primers |

| | |
|---|---|
| E5 | f: 5′AGCTGCTAGCGAAGTTTCTGCTTTGGAAAAGGAAGTTTCTGCTTTGGAAAAGGAAGTTTCTG |
| | CCTTAGAGAAAGAGGTCTCC-3′ |
| | (SEQ ID NO: 28) |
| | r: 5′CACCACTAGTCTTTTCCAAAGCAGAAACTTCCTTTTCCAAAGCGGAGACCTCTTTCTCTAAGG |
| | C-3′ |
| | (SEQ ID NO: 29) |
| K5 | f: 5′CACCATCGATTTCCTTCAAAGCAGAAACCTTTTCCTTCAAAGCAGAAACCTTTTCCTTCAAAG |
| | CGGAGACTTTCTCTTTTAATGCACT-3′ |
| | (SEQ ID NO: 30) |
| | r: 5′CTCGACGCGTGGTGGTGGTGGTTCTGGTGGTGGTGGTTCTAAGGTTTCTGCTTTGAAGGAAA |
| | AGGTTAGTGCATTAAAAGAGAAAGTCTCC-3′ |
| | (SEQ ID NO: 31) |
| LFA-1 1 | f: 5′-TTGTGTCGACAACGTAGACCTGGTATTTCT-3′ |
| domain | (SEQ ID NO: 32) |
| | r: 5′-CCATGGAGTCAGGTCCTGTTTGCTTG-3′ |
| | (SEQ ID NO: 33) |
| AL57 | f: 5-GGATGCTAGCGAAGTTCAATTGTTAGAGTC-3′ |
| | (SEQ ID NO: 34) |
| | r: 5′-GGTCGGATCCAGTTCGTTTGATTTCCACC-3′ |
| | (SEQ ID NO: 35) |
| TS1/22 | f: 5′-AGCAGCTAGCCAGGTTCACCTGCAGCAATC-3′ |
| | (SEQ ID NO: 36) |
| | r: 5′-CACCACTAGTAGCCCGTTTCAGCTCCAGC-3′ |
| | (SEQ ID NO: 37) |
| BoNT/A-LC | f: 5′-CGTTGTCGACCAATTCGTTAACAAGCAGTTCAAC-3′ |
| | ((SEQ ID NO: 38) |
| | r: 5′-AGTCCCATGGTTTGCTCGTTATAATGCCTCTAAC-3′ |
| | (SEQ ID NO: 39) |
| BoNT/B-LC | f: 5′-CGTTGTCGACCCGGTGACGATCAACAACTTC-3′ |
| | (SEQ ID NO: 40) |
| | r: 5′-AGTCCCATGGAACGCTCTTACACATTTGGATTT-3′ |
| | (SEQ ID NO: 41) |
| VHH-B8 & | f: 5-AGCTGCTAGCGCATGCCAGGCTCATGTCCAGCTGC-3′ |
| G6 | (SEQ ID NO: 42) |
| | r: 5′-GTTCGGATCCACTAGTTGGTTGTGGTTTTGGTGTCTTGG-3′ |
| | (SEQ ID NO: 43) |
| VHH | f: 5′-AGCTGCTAGCCAGRCTSAGKTGCAGCTCGTGGAG-3′ |
| library | (SEQ ID NO: 44) |
| | r-I: 5′-TGGTGGATCCTTGTGGTTTTGGTGTCTTGGG-3′ |
| | (SEQ ID NO: 45) |
| | r-II: 5′-TGGTGGATCCGGGGTCTTCGCTGTGGTGCG-3′ |
| | (SEQ ID NO: 46) |

| SEQ ID | Protein/Nucleotide | Description |
|---|---|---|
| 1 | amino acid | FLAG epitope |
| 2 | amino acid | Myc epitope |
| 3 | amino acid | HA epitope |
| 4 | amino acid | 6x His epitope |
| 5 | amino acid | T7 epitope tag |
| 6 | amino acid | the heptad unit of acid (En) coiled coils |
| 7 | amino acid | the heptad unit of base (Kn) coiled coils |
| 8 | nucleotide | acid E5 coiled coils |
| 9 | nucleotide | acid E4 coiled coils |
| 10 | nucleotide | acid E3 coiled coils |
| 11 | nucleotide | base K5 coiled coils |
| 12 | nucleotide | based K4 coiled coils |
| 13 | nucleotide | based K3 coiled coils |
| 14 | nucleotide | α1 mating type factor signal sequence |
| 15 | nucleotide | Aga2 signal sequence |
| 16 | amino acid | α1 mating type factor signal sequence |
| 17 | amino acid | Aga2 signal sequence |
| 18 | nucleotide | Aga2 |
| 19 | amino acid | Aga2 |
| 20 | nucleotide | NeGFP |
| 21 | amino acid | NeGFP |
| 22 | nucleotide | CeGFP |
| 23 | amino acid | CeGFP |
| 24 | nucleotide | GAL1-GAL10 bi-directional promoter |
| 25 | nucleotide | GAL1 promoter |
| 26 | nucleotide | GAL10 promoter |
| 27 | amino acid | Glycine linker sequence |
| 28 | nucleotide | forward primer for E5 |
| 29 | nucleotide | reverse primer for E5 |
| 30 | nucleotide | forward primer for K5 |
| 31 | nucleotide | reverse primer for K5 |
| 32 | nucleotide | forward primer for LFA-1 I domain |
| 33 | nucleotide | reverse primer for LFA-1 I domain |
| 34 | nucleotide | forward primer for AL57 |
| 35 | nucleotide | reverse primer for AL57 |
| 36 | nucleotide | forward primer for TS1/22 |
| 37 | nucleotide | reverse primer for TS1/22 |
| 38 | nucleotide | forward primer for BoNT/A-LC |
| 39 | nucleotide | reverse primer for BoNT/A-LC |
| 40 | nucleotide | forward primer for BoNT/B-LC |
| 41 | nucleotide | reverse primer for BoNT/B-LC |
| 42 | nucleotide | forward primer for VHH-B8&G6 |
| 43 | nucleotide | reverse primer for VHH-B8&G6 |
| 44 | nucleotide | forward primer for VHH library |
| 45 | nucleotide | reverse primer I for VHH library |
| 46 | nucleotide | reverse primer II for VHH library |
| 47 | amino acid | VHH1a |
| 48 | amino acid | VHH48 |
| 49 | amino acid | VHH57 |
| 50 | amino acid | VHH41 |
| 51 | amino acid | G6 |
| 52 | amino acid | B8 |
| 53 | amino acid | B10 |
| 54 | amino acid | C3 |

### REFERENCES

1. Fields, S., and Song, O. (1989) Nature 340(6230), 245-246
2. Ghosh, I., Hamilton, A. D., and Regan, L. (2000) Journal of the American Chemical Society 122(23), 5658-5659
3. Hu, C. D., Chinenov, Y., and Kerppola, T. K. (2002) Mol Cell 9(4), 789-798
4. Magliery, T. J., Wilson, C. G., Pan, W., Mishler, D., Ghosh, I., Hamilton, A. D., and Regan, L. (2005) J Am Chem Soc 127(1), 146-157
5. Remy, I., and Michnick, S. W. (2004) Methods Mol Biol 261, 411-426
6. Tarassov, K., Messier, V., Landry, C. R., Radinovic, S., Molina, M. M. S., Shames, I., Malitskaya, Y., Vogel, J., Bussey, H., and Michnick, S. W. (2008) Science 320(5882), 1465-1470
7. Stagljar, I., and Fields, S. (2002) Trends Biochem Sci 27(11), 559-563
8. Nyfeler, B., Michnick, S. W., and Hauri, H. P. (2005) Proc Natl Acad Sci USA 102(18), 6350-6355
9. Arnon, S. S., Schechter, R., Inglesby, T. V., Henderson, D. A., Bartlett, J. G., Ascher, M. S., Eitzen, E., Fine, A. D., Hauer, J., Layton, M., Lillibridge, S., Osterholm, M. T., O'Toole, T., Parker, G., Perl, T. M., Russell, P. K., Swerdlow, D. L., and Tonat, K. (2001) Jama-Journal of the American Medical Association 285(8), 1059-1070
10. Jin, M., Song, G., Carman, C. V., Kim, Y. S., Astrof, N. S., Shimaoka, M., Wittrup, D. K., and Springer, T. A. (2006) Proc Natl Acad Sci USA 103(15), 5758-5763
11. Maass, D. R., Sepulveda, J., Pernthaner, A., and Shoemaker, C. B. (2007) J Immunol Methods 324(1-2), 13-25
12. Gietz, R. D., and Schiestl, R. H. (2007) Nat Protoc 2(1), 38-41
13. Colby, D. W., Kellogg, B. A., Graff, C. P., Yeung, Y. A., Swers, J. S., and Wittrup, K. D. (2004) Methods Enzymol 388, 348-358
14. Boder, E. T., and Wittrup, K. D. (1997) Nat Biotechnol 15(6), 553-557
15. Johnston, M., and Davis, R. W. (1984) Mol Cell Biol 4(8), 1440-1448
16. Egel-Mitani, M., Hansen, M. T., Norris, K., Snel, L., and Fiil, N. P. (1988) Gene 73(1), 113-120
17. De Crescenzo, G., Litowski, J. R., Hodges, R. S., and O'Connor-McCourt, M. D. (2003) Biochemistry 42(6), 1754-1763
18. Shimaoka, M., Xiao, T., Liu, J. H., Yang, Y., Dong, Y., Jun, C. D., McCormack, A., Zhang, R., Joachimiak, A., Takagi, J., Wang, J. H., and Springer, T. A. (2003) Cell 112(1), 99-111
19. Sanchez-Madrid, F., Krensky, A. M., Ware, C. F., Robbins, E., Strominger, J. L., Burakoff, S. J., and Springer, T. A. (1982) Proc Natl Acad Sci USA 79(23), 7489-7493
20. Huang, L., Shimaoka, M., Rondon, I. J., Roy, I., Chang, Q., Po, M., Dransfield, D. T., Ladner, R. C., Edge, A. S., Salas, A., Wood, C. R., Springer, T. A., and Cohen, E. H. (2006) J Leukoc Biol 80(4), 905-914
21. Hagihara, Y., and Kim, P. S. (2002) Proc Natl Acad Sci USA 99(10), 6619-6624
22. Shimaoka, M., Kim, M., Cohen, E. H., Yang, W., Astrof, N., Peer, D., Salas, A., Ferrand, A., and Springer, T. A. (2006) Proc Natl Acad Sci USA 103(38), 13991-13996
23. Hamers-Casterman, C., Atarhouch, T., Muyldermans, S., Robinson, G., Hamers, C., Songa, E. B., Bendahman, N., and Hamers, R. (1993) Nature 363(6428), 446-448
24. Jeong, K. J., Seo, M. J., Iverson, B. L., and Georgiou, G. (2007) Proc Natl Acad Sci USA 104(20), 8247-8252
25. Dranginis, A. M., Rauceo, J. M., Coronado, J. E., and Lipke, P. N. (2007) Microbiology and Molecular Biology Reviews 71(2), 282-+
26. Hanes, J., and Pluckthun, A. (1997) Proc Natl Acad Sci USA 94(10), 4937-4942
27. Smith, G. P. (1985) Science 228(4705), 1315-1317
28. Krebber, C., Spada, S., Desplancq, D., Krebber, A., Ge, L., and Pluckthun, A. (1997) J Mol Biol 268(3), 607-618
29. Boder, E. T., Bill, J. R., Nields, A. W., Marrack, P. C., and Kappler, J. W. (2005) Biotechnol Bioeng 92(4), 485-491
30. van den Beucken, T., Pieters, H., Steukers, M., van der Vaart, M., Ladner, R. C., Hoogenboom, H. R., and Hufton, S. E. (2003) FEBS Lett 546(2-3), 288-294
31. Jin, M., Andricioaei, I., and Springer, T. A. (2004) Structure 12(12), 2137-2147
32. Appl Environ Microbiol. 2002 September; 68(9):4517-22
33. Raemarkers, R. J., de Muro, L., Gatehouse, J. A., Fordham-Skelton, A. P. (1999). Eur. J. Biochem. 265, 394-403.

What is claimed is:

1. A yeast cell, comprising two expression cassettes, wherein the first expression cassette comprises from 5′ to 3′: a first promoter, a nucleotide sequence coding for a fusion protein between a bait protein and a yeast cell wall anchor protein wherein the fusion protein comprises a first signal sequence, and a first 3′ untranslated region; the second expression cassette comprises from 5′ to 3′: a second promoter, a nucleotide sequence coding for a prey protein in fusion to a second signal sequence, and a second 3′ untranslated region; and wherein upon expression of the bait and the prey protein in said yeast cell, said bait protein becomes anchored in the cell wall, and said prey protein is produced as a secretory protein.

2. The yeast cell of claim 1, wherein said first and second promoters are identical inducible promoters functional in said yeast cell.

3. The yeast cell of claim 1, wherein said first promoter is one of the GAL1 or GAL10 promoter of S. cerevisiae, and the second promoter is the other of the GAL1 or GAL10 promoter of S. cerevisiae.

4. The yeast cell of claim 1, wherein said cell wall anchor protein is selected from the Aga2 protein or the flocculation domain of the flocculation protein (Flo1p).

5. The yeast cell of claim 1, wherein said first and second signal sequences are independently selected from the signal sequences of the Aga2 protein, α-1 mating factor, PHA (phytohemagglutinin), and the flocculation protein (Flo1p).

6. The yeast cell of claim 1, wherein said bait protein is linked to a first reporter molecule, and/or said prey protein is linked to a second reporter molecule.

7. The yeast cell of claim 6, wherein said bait protein is linked to a first reporter molecule, said prey protein is linked to a second reporter molecule, and said first and second reporter molecules are different epitope tags.

8. The yeast cell of claim 7, wherein said epitope tags are selected from Myc, FLAG, HA, 6xHis, and T7 epitopes.

9. The yeast cell of claim 6, wherein said bait protein is linked to a first reporter molecule, said prey protein is linked to a second reporter molecule, and said first and second reporter molecules are complementation fragments of a protein which, upon binding to each other, reconstitute the protein.

10. The yeast cell of claim 9, wherein said protein is selected from the group consisting of green fluorescent protein (GFP), β-lactamase, and dihydrofolate reductase (DHFR).

11. The yeast cell of claim 1, wherein said bait protein is an antibody, and said prey protein is an antigen.

12. The yeast cell of claim 1, where said yeast is selected from the group consisting of Saccharomyces cerevisiae, Schizosaccharomyces pombe, Pichia pastoris, and species of the Candida genus.

13. The yeast cell of claim 1, wherein the two expression cassettes are provided in a single vector.

14. A library of yeast cells, wherein each cell in the library comprises two expression cassettes, wherein the first expression cassette comprises from 5′ to a first promoter, a nucleotide sequence coding for a fusion protein between a bait protein and a yeast cell wall anchor protein wherein the fusion protein comprises a first signal sequence, and a first 3′ untranslated region; the second expression cassette comprises from 5′ to 3′: a second promoter, a nucleotide sequence coding for a prey protein in fusion to a second signal sequence, and a second 3′ untranslated region; and wherein upon expression of the bait and the prey protein in said yeast cell, said bait protein becomes anchored in the cell wall, and said prey protein is produced as a secretory protein; and wherein (1) the bait protein is the same for all of the cells in the library, and the prey proteins are different for all or at least some of the cells in the library; (2) the prey protein is the same for all of the cells in the library, and the bait proteins are different for all or at least some of the cells in the library; or (3) the bait and the prey proteins are each different for all or at least some of the cells in the library.

15. The library of claim 14, wherein said bait protein is linked to a first reporter molecule, and said prey protein is linked to a second reporter molecule.

16. A yeast surface two hybrid (YS2H) system, comprising:
a yeast cell,
a vector comprising two expression cassettes, wherein the first expression cassette comprises from 5′ to 3′: a first promoter, a nucleotide sequence coding for a fusion protein between a bait protein and a yeast cell wall anchor protein wherein the fusion protein comprises a first signal sequence, and a first 3′ untranslated region; the second expression cassette comprises from 5′ to 3′: a second promoter, a nucleotide sequence coding for a prey protein in fusion to a second signal sequence, and a second 3′ untranslated region; and wherein upon expression of the bait and the prey protein in said yeast cell, said bait protein becomes anchored in the cell wall, and said prey protein is produced as a secretory protein; and
reagents or instructions for detecting signals generated upon association of the bait and the prey proteins on the cell surface of said yeast cell.

17. The system of claim 16, wherein said bait protein is linked to a first reporter molecule, and said prey protein is linked to a second reporter molecule.

18. A method for assessing the interactions between two proteins, comprising:
providing a yeast cell,
providing two expression cassettes, wherein the first expression cassette comprises from 5′ to 3′: a first promoter, a nucleotide sequence coding for a fusion protein between a bait protein and a yeast cell wall anchor protein wherein the fusion protein comprises a first signal sequence, and a first 3′ untranslated region; the second expression cassette comprises from 5′ to 3′: a second promoter, a nucleotide sequence coding for a prey protein in fusion to a second signal sequence, and a second 3′ untranslated region; and wherein upon expression of the bait and the prey protein in said yeast cell, said bait protein becomes anchored in the cell wall, and said prey protein is produced as a secretory protein;
introducing said two expression cassettes into said yeast cell by transformation;
expressing said two proteins in said yeast cell to allow interactions between said two proteins; and
detecting signals generated upon association of the bait and the prey proteins on the cell surface of said yeast cell as a basis for assessing the interactions between the two proteins.

19. The method of claim 18, wherein said bait protein is linked to a first reporter molecule, and said prey protein is linked to a second reporter molecule.

20. The method of claim 19, wherein said assessing the interactions between the two proteins comprises assessing the binding affinity between the two proteins.

21. A method of identifying a candidate protein which binds to a target protein from a plurality of candidate proteins, comprising:
providing a library of yeast cells, wherein each cell in the library comprises two expression cassettes, wherein the first expression cassette comprises from 5′ to 3′: a first promoter, a nucleotide sequence coding for a fusion protein between said target protein as a bait protein and a yeast cell wall anchor protein wherein said fusion protein comprises a first signal sequence, and a first 3′ untranslated region; the second expression cassette comprises from 5′ to 3′: a second promoter, a nucleotide sequence coding for a candidate protein as a prey protein in fusion to a second signal sequence, and a second 3′ untranslated region; wherein upon expression of the bait and the prey protein in said yeast cell, said bait protein becomes anchored in the cell wall, and said prey protein is produced as a secretory protein; and wherein the first expression cassettes are identical among all of the cells in the library, and wherein the second expression cassettes are identical among the cells except for the candidate proteins such that a plurality of candidate proteins are represented by said library;
expressing said proteins to allow association between candidate proteins to said target protein;
detecting signals generated upon association of candidate proteins to said target protein as a basis for assessing the binding interactions between a candidate protein to said target protein;
sorting cells in said library based on binding affinities of candidate proteins to said target protein; and
identifying a cell expressing a candidate protein having a selected binding affinity to said target protein.

22. A method of identifying a candidate protein which binds to a target protein from a plurality of candidate proteins, comprising:
providing a library of yeast cells, wherein each cell in the library comprises two expression cassettes, wherein the first expression cassette comprises from 5′ to 3′: a first promoter, a nucleotide sequence coding for a fusion protein between a candidate protein as a bait protein and a yeast cell wall anchor protein wherein said fusion protein comprises a first signal sequence, and a first 3′ untranslated region; the second expression cassette comprises from 5′ to 3′: a second promoter, a nucleotide sequence coding for said target protein as a prey protein in fusion to a second signal sequence, and a second 3′ untranslated region; wherein upon expression of the bait and the prey protein in said yeast cell, said bait protein becomes anchored in the cell wall, and said prey protein is produced as a secretory protein; and wherein the second expression cassettes are identical among all of the cells in the library, and wherein the first expression cassettes are identical among the cells except for the candidate proteins such that a plurality of candidate proteins are represented by said library;
expressing said proteins to allow association between candidate proteins to said target protein;
detecting signals generated upon association of candidate proteins to said target protein as a basis for assessing the binding interactions between a candidate protein to said target protein;
sorting cells in said library based on binding affinities of candidate proteins to said target protein; and
identifying a cell expressing a candidate protein having a selected binding affinity to said target protein.

23. The method of claim 21 or 22, wherein said target protein is an antigen, and said candidate protein is an antibody.

24. A method of identifying interacting protein pairs, comprising:
providing a library of yeast cells, wherein each cell in the library comprises two expression cassettes, wherein the first expression cassette comprises from 5′ to 3′: a first promoter, a nucleotide sequence coding for a fusion protein between a candidate protein as a bait protein and a yeast cell wall anchor protein wherein said fusion protein comprises a first signal sequence, and a first 3′ untranslated region; the second expression cassette comprises from 5′ to 3′: a second promoter, a nucleotide sequence coding for a candidate binding partner protein as a prey protein in fusion to a second signal sequence, and a second 3′ untranslated region; wherein upon expression of the bait and the prey protein in said yeast cell, said bait protein becomes anchored in the cell wall, and said prey protein is produced as a secretory protein; and wherein the first expression cassettes are identical among the cells except for the candidate proteins such that a plurality of candidate proteins are represented by said library, and the second expression cassettes are identical among the cells except for the candidate binding partner proteins such that a plurality of candidate binding partner proteins are represented by said library;
expressing said proteins to allow association between candidate proteins to said candidate binding partner proteins;
detecting signals generated upon association of candidate proteins to said candidate binding partner proteins;
sorting cells in said library based on binding affinities of candidate proteins to said candidate binding partner proteins; and
identifying a cell expressing a candidate protein and a candidate binding partner protein having a selected binding affinity towards each other.
